(19) United States
(12) Patent Application Publication
(10) Pub. No.: US 2016/0193213 A1
(43) Pub. Date: Jul. 7, 2016

(54) PHARMACEUTICAL FORMULATIONS OF XANTHINE OR XANTHINE DERIVATIVES

(71) Applicant: Imprimis Pharmaceuticals, Inc., San Diego, CA (US)

(72) Inventor: Mark L. Baum, San Diego, CA (US)

(21) Appl. No.: 14/982,322

(22) Filed: Dec. 29, 2015

Related U.S. Application Data
(60) Provisional application No. 62/100,368, filed on Jan. 6, 2015.

(51) Int. Cl. A61K 31/522; A61K 45/06; A61K 9/00

(52) U.S. Cl. CPC A61K 31/522; A61K 9/0014; A61K 45/06

(57) ABSTRACT

Pharmaceutical compositions and methods for treating various diseases and pathologies, such as Dupuytren contracture, are described, the compositions comprising pentoxifylline and a pharmaceutically acceptable carrier. Methods for fabricating the compositions and using them are also described.

### CROSS REFERENCE TO RELATED APPLICATIONS

This application claims priority under 35 U.S.C. §119(e) to U.S. Provisional Application No. 62/100,368 filed on Jan. 6, 2015, entitled “Pharmaceutical Formulations of Xanthine or Xanthine Derivatives,” the entire content of which is hereby incorporated by reference.

### FIELD OF THE INVENTION

The present invention relates generally to the field of pharmacology and more specifically to compositions and methods designed to treat, mitigate or prevent various diseases and pathologies, such as Dupuytren's contracture, and to methods of preparing and using such compositions.

### BACKGROUND

The present disclosure relates to pharmaceutical formulations comprising xanthine or a xanthine derivative, such as pentoxifylline, and methods for treating various diseases and pathologies (e.g., Dupuytren's contracture) by local administration.

Fibrotic diseases can be found in a variety of tissues. For example, Dupuytren's contracture, also described as Dupuytren's disease or morbus Dupuytren, is believed to be caused by fibromatosis of the palm. Clinically, this usually leads to flexion contracture and manifests itself as involuntary “clawing” of the hand, i.e., a painful situation when the fingers tend to bend inwardly towards the center of the palm and cannot be easily and painlessly straightened. Painful nodules and cords are often formed in the hand as the disease progresses.

Various methods and therapies have been suggested for the treatment of Dupuytren's disease and related disorders that are mentioned below. In particular, oral administration of a nonspecific phosphodiesterase inhibitors (PDEi) has been suggested and tried, but no more than minimal to moderate improvement has been achieved by such methods. Therefore, there remains a need for better treatments of such diseases.

This patent specification discloses such pharmaceutical compositions suitable for treatment and alleviation of various diseases and pathologies, including Dupuytren's disease, which can achieve positive patient outcomes while free of drawbacks and deficiencies of existing formulations, and methods of fabricating and administering the same.

### SUMMARY

According to one embodiment of the invention, there is provided a method for treating a disease, disorder or pathological condition, such as Dupuytren's contracture, frozen shoulder, lipoma, cellulite, uterine fibroids, glaucoma, hyperthrophic scars, scarred tendons, keloids, herniated intervertebral disks or vitrectomy, in a mammalian subject in a need of the treatment. The method includes locally administering to the subject a pharmaceutical formulation comprising a therapeutically effective amount of a compound of formula I:

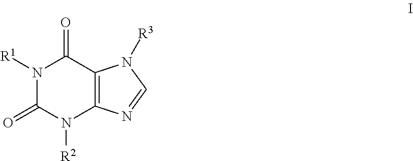

or a pharmaceutically acceptable salt, solvate or hydrate thereof, wherein each of R¹, R² and R³ is independently any of H, a C₁-C₆ alkyl, a C₂-C₆ alkenyl, a C₂-C₆ alkynyl, a cycloalkyl, a heterocyclyl, an aryl or a heteroaryl, each of which is further optionally substituted.

According to another embodiment of the invention, the compound of formula I is pentoxifylline: 3,7-dimethyl-1-(5-oxohexyl)-3,7-dihydro-1H-purine-2,6-dione or 1-(5-oxohexyl)-3,7-dimethylxantine.

### DETAILED DESCRIPTION

### A. Terms and Definitions

Unless specific definitions are provided, the nomenclatures utilized in connection with, and the laboratory procedures and techniques of analytical chemistry, synthetic organic and inorganic chemistry described herein, are those known in the art. Standard chemical symbols are used interchangeably with the full names represented by such symbols. Thus, for example, the terms “hydrogen” and “H” are understood to have identical meaning. Standard techniques may be used for chemical syntheses, chemical analyses, formulating compositions and testing them. The foregoing techniques and procedures can be generally performed according to conventional methods well known in the art.

It is to be understood that both the foregoing general description and the following detailed description are exemplary and explanatory only and are not restrictive of the invention claimed. As used herein, the use of the singular includes the plural unless specifically stated otherwise. The section headings used herein are for organizational purposes only and are not to be construed as limiting the subject matter described.

As used herein, “or” means “and/or” unless stated otherwise. Furthermore, use of the term “including” as well as other forms, such as “includes,” and “included,” is not limiting.

“About” as used herein means that a number referred to as “about” comprises the recited number plus or minus 1-10% of that recited number. For example, “about” 100 degrees can mean 95-105 degrees or as few as 99-101 degrees depending on the context. Whenever it appears herein, a numerical range such as “1 to 20” refers to each integer in the given range; i.e., meaning only 1, only 2, only 3, etc., up to and including only 20.

The term “pharmaceutical composition” is defined as a chemical or biological compound or substance, or a mixture or combination of two or more such compounds or substances, intended for use in the medical diagnosis, cure, treatment, or prevention of disease or pathology.

The terms “Dupuytren's contracture” and “Dupuytren's disease,” used herein interchangeably, are defined as one or several conditions associated with, or caused by, a proliferative connective tissue disorder in the hand's palmar fascia and which manifests itself as a flexion contracture of the hand, typically due to a palmar fibromatosis. As the disease progresses, the fingers tend to bend inwardly towards the palm and cannot be fully and/or painlessly extended.

The term “frozen shoulder” is defined as one or several conditions associated with, or caused by, the inflammation of shoulder capsule, which is the connective tissue surrounding the glenohumeral joint of the shoulder.

The term “lipoma” is defined as a benign tumor formed by a fatty tissue on various parts of a body.

The term “cellulite” is defined as formation of protrusions of subcutaneous fat within fibrous connective tissue typically on the buttocks or abdomen of a patient.

The term “uterine fibroids” is defined as benign tumors that develop in the uterus of a female patient.

The term “glaucoma” is defined as one or several conditions associated with, or caused by, damage to the optic nerve due to increased intraocular pressure.

The term “hyperthrophic scars” is defined as a skin condition typically developing after a thermal or traumatic injury and characterized by the resulting scar to be raised above the surrounding skin.

The term “keloids” is defined as benign scars comprised of fibrous nodules which are formed by excessive deposits of collagen on a patient's skin.

The term “herniated intervertebral disks” refers to a medical condition in which a tear in the fibrous ring of an intervertebral disc causes the cushion that sits between the spinal vertebra to be pushed outside its normal position.

The term “vitrectomy” is defined a surgical procedure to remove some or all of the vitreous humor from the eye of a patient.

The terms “solvate” and “hydrate” are used herein to indicate that a compound or substance is physically or chemically associated with a solvent for “solvates” such as water (for “hydrates”).

The term “carrier” refers to a substance that serves as a vehicle for improving the efficiency of delivery and the effectiveness of a pharmaceutical composition.

The term “excipient” refers to a pharmacologically inactive substance that is formulated in combination with the pharmacologically active ingredient of pharmaceutical composition and is inclusive of bulking agents, fillers, diluents and products used for facilitating drug absorption or solubility or for other pharmacokinetic considerations.

The term “mono therapy” as used herein refers to a method of treatment where only one therapeutic or pharmacologically active agent is utilized; the “combo therapy” involves the use of at least two such agents.

The term “therapeutically effective amount” is defined as the amount of the compound or pharmaceutical composition that will elicit the biological or medical response of a tissue, system, animal or human that is being sought by the researcher, medical doctor or other clinician.

The term “pharmaceutically acceptable” is defined as a carrier, whether diluent or excipient, that is compatible with the other ingredients of the formulation and not deleterious to the recipient thereof.

The terms “administration of a composition” or “administering a composition” is defined to include an act of providing a compound of the invention or pharmaceutical composition to the subject in need of treatment.

The terms “local administration” and “locally administering” as used herein refer to treatment of a fibrotic disease by administering at sites approximate to local symptoms (e.g., Dupuytren cords) of the fibrotic disease. It is distinguished from systemic administrations, such as oral administration or intravenous injection, wherein dosage of a pharmaceutical composition is relatively similar throughout the body of a subject. Non-limiting examples of local administration include injection injection into a palpable cord, topical administration, and transdermal administration.

### B. Embodiments of the Invention

According to embodiments of the present invention, there are provided methods for treating several diseases, disorders or pathological conditions such as Dupuytren's contracture, frozen shoulder, lipoma, cellulite, uterine fibroids, glaucoma, hyperthrophic scars, scarred tendons, keloids, herniated intervertebral disks or vitrectomy, in a mammalian subject in a need of the treatment. The methods include administering to the subject a pharmaceutical formulation comprising a therapeutically effective amount of a tumor necrosis factor (TNF) antagonist or inhibitor such as a compound of formula I:

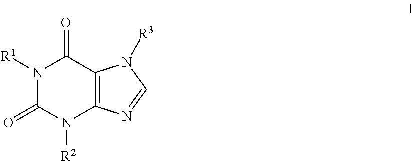

or a pharmaceutically acceptable salt, solvate or hydrate thereof, wherein each of R¹, R² and R³ is independently any of H, a C₁-C₆ alkyl, a C₂-C₆ alkenyl, a C₂-C₆ alkynyl, a cycloalkyl, a heterocyclyl, an aryl or a heteroaryl, each of which may be further optionally substituted. The composition may include a single compound of formula I or a combination of several such compounds each of which is described by formula I. In some embodiments, each of R¹, R² and R³ is independently any of H, or a C₁-C₆ alkyl optionally substituted with a hydroxyl or acyl group (carbonyl or aldehyde).

The quantity of compound of formula I in the pharmaceutical formulation expressed as molar concentration can be between about 0.03 mM and about 3 mM of compound of formula I per 1 μL of the entire formulation. In some embodiments, the therapeutic effective amount of compound of formula I in the pharmaceutical formulation is between about 0.1 mg and about 20 mg such as between about 0.3 mg and about 10 mg, for example, about 0.5 mg, or about 4 to about 20 mg.

In one embodiment the compound of formula I is a nonspecific phosphodiesterase inhibitor (PDEi) such as pentoxifylline, i.e., 1-(5-oxohexyl)-3,7-dimethylxanthine, i.e., a compound formula I where each of R² and R³ is methyl and R¹ is 5-oxohexyl, i.e., a functional group having the structure —(CH₂)₄—C(O)—CH₃. Lisofylline, an active metabolite of pentoxifylline, i.e., 1-(5-hydroxyhexyl)-3,7-dimethylxanthine can be also used if desired. The structure of lisofylline is basically the same as that of pentoxifylline except its functional group R¹ includes a primary alcohol moiety —C(OH)— instead of the acyl moiety —C(O)— that is present in the R¹ group in pentoxifylline. Other non-limiting examples of compounds encompassed by formula I that can be used include caffeine, aminophylline (theophylline with ethylenediamine), enprofylline (3-propylxantine), isbufylline (1,3-dimethyl-7-isobutylxantine), theophylline, theobromine, 3-isobutyl-1-methylxanthine, oxitriphylline (choline theophyllinate), dyphylline (diprophylline or 7-(2,3-dihydroxypropyl)-1,3-dimethyl-3,7-dihydro-1H-purine-2,6-dione); 1-(5-hydroxy-5-methylhexyl)-3-methylxanthine (albifylline); 7-ethoxymethyl-1-(5-hydroxy-5-methylhexyl)-3-methylxanthine (torbafylline); and 7-propyl-1-(5-hydroxy-5-methylhexyl)-3-methylxanthine.

In some embodiments, provided herein is a method for treating a fibrotic disease in a subject in need thereof, which comprises locally administering to the subject a pharmaceutical formulation comprising, consisting essentially of, or consisting of, a therapeutic effective amount of a nonspecific PDEi or a pharmaceutically acceptable salt thereof, wherein the nonspecific PDEi is pentoxifylline, caffeine, aminophylline, enprofylline, isbufylline, theophylline, theobromine or 3-isobutyl-1-methylxanthine.

In some embodiments, the pharmaceutical formulation further comprises a pharmaceutically acceptable excipient or carrier, including, but not limited to, an antioxidant, an adjuvant or synergist, and a preservative.

Non-limiting examples of the antioxidant that can be used include α-tocopherol acetate, acetone sodium bisulfite, acetylcysteine, ascorbic acid, ascorbyl palmitate, butylated hydroxyanisole, butylated hydroxytoluene, cysteine, cysteine hydrochloride, d-α-tocopherol natural, d-α-tocopherol synthetic, dithiothreitol, monothioglycerol, nordihydroguaiaretic acid, propyl gallate, sodium bisulfite, sodium formaldehyde sulfoxylate, sodium metabisulfite, sodium sulfite, sodium thiosulfate, thiourea, and tocopherols.

Non-limiting examples of the adjuvant or synergist include citric acid, EDTA (ethylenediaminetetra acetic acid), its conjugate base, and salts, hydroxyquinoline sulfate, phosphoric acid, and tartaric acid.

In those embodiments where the formulation includes an EDTA sodium salt as an adjuvant, the EDTA sodium salt can be 0-0.15% by weight of the formulation, for example, 0.01, 0.02, 0.03, 0.04, 0.05, 0.06, 0.07, 0.08, 0.09, 0.10, 0.11, 0.12, 0.13, 0.14, or 0.15% by weight of the formulation. If an EDTA magnesium salt is used as an adjuvant, the EDTA magnesium salt can be 0-0.15% by weight of the formulation, for example, 0.01, 0.02, 0.03, 0.04, 0.05, 0.06, 0.07, 0.08, 0.09, 0.10, 0.11, 0.12, 0.13, 0.14, or 0.15% by weight of the formulation.

Non-limiting examples of the preservative are benzalkonium chloride, benzethonium chloride, benzoic acid and salts, benzyl alcohol, boric acid and salts, cetylpyridinium chloride, cetyltrimethyl ammonium bromide, chlorobutanol, chlorocresol, chorhexidine gluconate or chlorhexidine acetate, cresol, ethanol, imidazolidinyl urea, metacresol, methylparaben, nitromersol, o-phenyl phenol, parabens, phenol, phenylmercuric acetate/nitrate, propylparaben, sodium benzoate, sorbic acids and salts, β-phenylethyl alcohol, and thimerosal. In particular embodiments, the preservative is benzyl alcohol.

In those embodiments where the formulation includes ethanol as a preservative, ethanol can be 190 proof. The ethanol can be 0-15% by volume of the formulation, for example, 0, 1, 2, 3, 4, 5, 6, 7, 8, 9, 10, 11, 12, 13, 14, or 15% by volume of the formulation. In those embodiments where the formulation includes benzyl alcohol as a preservative, the benzyl alcohol can be 0-1.5% by weight of the formulation, for example, 0, 0.1, 0.2, 0.3, 0.4, 0.5, 0.6, 0.7, 0.8, 0.9, 1.0, 1.1, 1.2, 1.3, 1.4, or 1.5% by volume of the formulation.

In some embodiments, the pharmaceutical formulation is filtered before local administration. In particular embodiments, the pharmaceutical formulation is filtered through a 0.22 micron filter before local administration. In other embodiments, the pharmaceutical formulation has a pH of between 4 and 8. In particular embodiments, the pharmaceutical formulation has a pH of between 5.5 and 6. The pH can be adjusted by adding acids or bases, e.g., HCl or NaOH.

The pharmaceutical formulation can be administered to a subject in need thereof by various local administrations, e.g., by injection one to four times in a twenty-four hour period. In particular embodiments, the pharmaceutical formulation is administered daily until desired effects are achieved.

In certain embodiments, the pharmaceutical formulation is administered topically. In other embodiments, the pharmaceutical formulation is administered transdermally. In still other embodiment, the pharmaceutical formulation is administered locally by injection directly into the area of the fibrotic disease. In particular embodiments, in case of treatment of the Dupuytren contracture, for example, the pharmaceutical formulation is injected directly into Dupuytren cord(s).

In certain embodiments, the pharmaceutical formulation further comprises one or more additional active agent(s). In particular embodiments, the second active agent is a vasodilator, e.g., alprostadil (prostaglandin E₁), papaverine, phentolamine, α-receptor blocking agents, ergot alkaloids, antihypertensive agents, vasodilators, nitrovasodilators, naturally occurring, semisynthetic and synthetic prostaglandins, and/or vasoactive intestinal peptide. In other embodiments, the pharmaceutical formulation further comprises a collagenase, such as collagenase clostridium histolyticum.

The methods provided herein can be used as a mono therapy or a part of a combo therapy. In certain embodiments, the formulation comprising the compound of formula I, e.g., pentoxifylline, is used as a mono therapy. In certain particular embodiments, the formulation consisting essentially of a nonspecific PDEi, e.g., pentoxifylline, is used as a mono therapy to treat a fibrotic disease, such as Dupuytren's contracture.

In other embodiments, the formulation comprising the compound of formula I, e.g., pentoxifylline, is used as a part of a combo therapy, for example, when the formulation consisting essentially of a nonspecific PDEi, e.g., pentoxifylline, is used to treat a fibrotic disease, such as Dupuytren's disease, in combination with a collagenase therapy, e.g., collagenase clostridium histolyticum or Xiaflex® (collagenase clostridium histolyticum) from Auxilium Pharmaceuticals, Inc. of Chesterbrook, Pa.

The pharmaceutical formulations that are described herein may, in addition, optionally contain other pharmacologically active compounds, such as at least one anti-bacterial agent(s), or at least one antiviral medicament(s) and combinations thereof. Those having ordinary skill in the art can determine what specific anti-bacterial and/or antiviral medicament(s) are to be used, if any.

The concentration of the anti-bacterial agent(s) in the compositions of the present application may be between about 0.01 mg/mL and about 50.0 mg/mL, such as between about 0.5 mg/mL and about 10.0 mg/mL, for example, about 1.0 mg/mL. Non-limiting examples of the anti-bacterial agents that may be used include fluoroquinolones such as moxifloxacin, gatifloxacin, nalidixic acid, oxolinic acid, piromidic acid, pipemidic acid, rosoxacin, enoxacin, fleroxacin, lomefloxacin, nadifloxacin, ofloxacin, pefloxacin, rufloxacin, balofloxacin, levofloxacin, norfloxacin, ciprofloxacin, pazufloxacin, sparfloxacin, tosufloxacin, clinafloxacin, gemifloxacin, sitafloxacin, prulifloxacin and combinations thereof.

Non-limiting examples of anti-bacterial agents other than fluoroquinolones that may be used include vancomycin, teicoplanin, telavancin, decaplanin, ramoplanin, azitromycin, gentamicin, tobramycin, amikacin, cefuroxime, mitomycin, neomycin, neosporin, amoebicides (e.g., metronidazole, tinidazole, secnidazole, ornidazole, polyhexamethylene biguanide or chlorohexidine), polymyxin, clindamycin, bacitracin, chloramphenicol, erythromycin, natamycin, blephamide, sulfacetamide, sodium bicarbonate, povidone-iodine and combinations thereof.

The concentration of the antiviral medicament(s) in the compositions of the present application may be between about 0.01 mg/mL and about 75.0 mg/mL, such as between about 1 mg/mL and about 50.0 mg/mL, for example, about 20.0 mg/mL. Non-limiting examples of the antiviral medicaments that may be used include idoxuridine, vidarabine and combinations thereof.

As mentioned above, the pharmaceutical composition that is the subject matter of the instant application may further optionally include one or several pharmaceutically acceptable excipient(s). In some embodiments, an excipient that can be used may be a non-ionic polyoxyethylene-polyoxypropylene block copolymer having the following general structure:

HO—(CH₂—CH₂—O)ₓ—(C₃H₆—O)_{y}—(CH₂—CH₂—O)ₓ—H,

wherein x is an integer having the value of at least 8 and y is an integer having the value of at least 38.

If a non-ionic polyoxyethylene-polyoxypropylene block copolymer is used as an excipient, its contents in the overall composition may be between about 0.01 mass % and about 20.0 mass % such as between about 1.0 mass % and about 15 mass %, for example, about 10.0 mass %.

One non-limiting example of a specific non-ionic polyoxyethylene-polyoxypropylene block copolymer that can be used as a solubilizing and stabilizing agent in the pharmaceutical compositions of the instant invention is the product known under the trade name Poloxamer 407® (poly(ethylene glycol)-block-poly(propylene glycol)-block-poly(ethylene glycol)) available from Sigma-Aldrich Corp. of St. Louis, Mo., with the molecular weight of the polyoxypropylene portion of about 4,000 Daltons, about a 70% polyoxyethylene content, the overall molecular weight of between about 9,840 Daltons and about 14,600 Daltons, and having the following chemical structure:

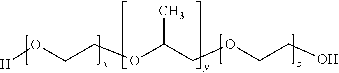

wherein x=z and each is between about 78 and about 116; y is about 69.

According to further embodiments, the excipient portion of the pharmaceutical formulation may contain other products, instead of, or in combination with, non-ionic polyoxyethylene-polyoxypropylene block copolymer(s). One non-limiting example of such additional excipient is poly(acrylic acid) in its various cross-linked or non-cross-linked versions, such as Carbomer 940® having a weight-average molecular weight of about 940 and available from Lubrizol Corp. of Wickliffe, Ohio. Another type of products that can be used in the excipient portion of the pharmaceutical formulation may be water-soluble methylcellulose and hydroxypropyl methylcellulose polymers, such as Methocel® family of products available from Dow Chemical Co. of Midland, Mich., for example, a hydroxypropyl methylcellulose product Methocel® E4M.

According to further embodiments, methods for fabricating the above-described pharmaceutical compositions are provided. A one-batch formulation method may be used, where the components of the pharmaceutical formulation can be combined in single container; the components may be added to the container simultaneously or consecutively. Alternatively, a two- or multiple-batch method(s) may be used if desired, where each component of the pharmaceutical formulation can be combined in separate container followed by combining the contents of each container.

In one exemplary, non-limiting procedure, a quantity of a tumor necrosis factor inhibitor such as pentoxifylline may be placed into a mixing container followed by adding a quantity of sterile water and a polymeric gel (e.g., a Poloxamer 407®-based gel); the mixture is stirred until a clear stable solution is obtained, allowing the formulation to remain closed system thus preventing contamination and the loss of sterility.

The resulting product may then be transferred into single dose vials, capped, sealed, autoclaved and shaken until cool. Finally, complete sterility and endotoxin removal may be performed on the product according to commonly used methods known to those having ordinary skill in the art. As mentioned above, in some embodiments, the pharmaceutical compositions can be used for topical administration such as compositions formulated and delivered to a patient as injections. The compositions may also contain some quantity of preservative(s) such as benzalkonium chloride, if desired.

In one exemplary, non-limiting embodiment, illustrating in general the method for treating Dupuytren contracture, the process of administering pharmaceutical compositions described herein may be as follows. The pharmaceutical composition can be injected into a palpable cord with a contracture of a metacarpophalangeal or a proximal interphalangeal joint, the dose of the active pharmaceutical agent in the composition being typically between about 0.4 and about 0.7 mg per injection. The injection can be followed by the finger extension procedure, and then the injection/finger extension cycle may be repeated after approximately 24 to 72 hours. Injections and finger extension procedures may be administered up to 3 times per cord at approximately 4-week intervals. A reasonably skilled practitioner can select the equipment to be used for injections. For example, a 27-gauge ½-inch needle may be used.

It will be understood by those having ordinary skill in the art that the specific dose level and frequency of dosage for any particular patient may be varied and will depend upon a variety of factors including the activity of the specific compound employed, the metabolic stability and length of action of that compound, the age, body weight, general health, gender, diet, and the severity of the particular condition being treated.

In additional embodiments, pharmaceutical kits are provided. The kit includes a sealed container approved for the storage of pharmaceutical compositions, the container containing one of the above-described pharmaceutical compositions and a device for locally administering the formulation (e.g., a syringe and a needle). An instruction for the use of the composition and the information about the composition are to be affixed to the container or otherwise enclosed with it.

The following examples are provided to further elucidate the advantages and features of the present invention, but are not intended to limit the scope of the invention. The examples are for the illustrative purposes only. USP pharmaceutical grade products were used in preparing the formulations described below.

### Example 1

### Preparing a Pharmaceutical Composition No. 1

A pharmaceutical composition may be prepared as described below. The following products can be used in the amounts and concentrations specified:

(a) about 20.0 g of aqueous solution of Poloxamer 407®, at a concentration of Poloxamer 407® of about 20.0 mass %;

(b) about 0.11 g of Carbomer 940 (a powder); and

(c) about 100.0 mL of sterile water for injection.

Poloxamer 407® and Carbomer 940® can be thoroughly mixed with water, until fully dissolved, the pH may be adjusted to about 5.5 using sodium hydroxide. The product can then be refrigerated overnight, placed into a vial and autoclaved followed by adding the preservative benzalkonium chloride (at about 1:10,000 mass ratio) to form a stock Poloxamer/Carbomer gel to be used in further steps. Next, the following products may be used in the amounts and concentrations specified:

(d) about 1.0 g of pentoxifylline, at a concentration of about 1.0%;

(e) about 90 mL of the Poloxamer/Carbomer gel obtained as described above; and

(f) about 9.0 mL of sterile water for injection.

Pentoxifylline may be combined with the gel and water and the final product can be transferred into dropper bottles (10 mL size), capped and sealed. The product should have an estimated shelf life of about 90 days when kept refrigerated.

### Example 2

### Preparing a Pharmaceutical Composition No. 2

A pharmaceutical composition may be prepared as described below. The following products can be used in the amounts and concentrations specified:

(a) about 0.4 g of Methocel® E4M (a powder);

(b) about 0.2 g of Carbomer 940® (a powder); and

(c) about 100.0 mL of sterile water for injection.

The Methocel® E4M and Carbomer 940® powders can be combined in a beaker, then water can be added to allow hydrating overnight to form a solution, the pH may be adjusted to about 5.0 using sodium hydroxide. The gel can be autoclaved and cooled followed by adding preservative benzalkonium chloride (at about 1:10,000 mass ratio) to form a stock Methocel® E4M/Carbomer solution to be used in further steps. Next, the following products may be used in the amounts and concentrations specified:

(d) about 1.0 g of pentoxifylline, at a concentration of about 1.0%;

(e) about 90 mL of the Methocel® E4M/Carbomer solution obtained as described above; and

(f) about 9.0 mL of sterile water for injection.

Pentoxifylline may be combined with the gel and water and the final product can be transferred into dropper bottles (10 mL size), capped and sealed. The product should have an estimated shelf life of about 90 days when kept refrigerated.

Although the invention has been described with reference to the above examples, it will be understood that modifications and variations are encompassed within the spirit and scope of the invention. Accordingly, the invention is limited only by the following claims.

What is claimed is:

1. A method for treating a disease, disorder or pathological condition in a mammalian subject in need thereof, comprising locally administering to the subject a pharmaceutical formulation comprising a therapeutic effective amount of a compound of formula I: 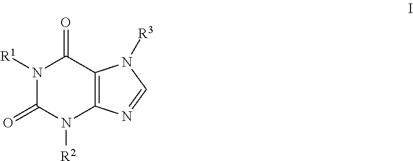
or a pharmaceutically acceptable salt thereof, wherein:
each of R¹, R² and R³ is independently selected from the group consisting of H, C₁-C₆ alkyl, C₂-C₆ alkenyl, C₂-C₆ alkynyl, cycloalkyl, heterocyclyl, aryl and heteroaryl, each of which is further optionally substituted,
with the further proviso that the disease, disorder or pathological condition is selected from the group consisting of Dupuytren's contracture, frozen shoulder, lipoma, cellulite, uterine fibroids, glaucoma, hyperthrophic scars, scarred tendons, keloids, herniated intervertebral disks and vitrectomy.

2. The method of claim 1, wherein each of R¹, R² and R³ is independently selected from the group consisting of H and an optionally substituted C₁-C₆ alkyl.

3. The method of claim 1, wherein each of R¹, R² and R³ is independently selected from the group consisting of H, a C₁-C₆ alkyl and an acyl-substituted C₁-C₆ alkyl.

4. The method of claim 1, wherein the compound of formula I is a nonspecific phosphodiesterase inhibitor.

5. The method of claim 4, wherein the nonspecific phosphodiesterase inhibitor is selected from the group consisting of pentoxifylline, caffeine, aminophylline, enprofylline, isbufylline, theophylline, theobromine and 3-isobutyl-1-methylxanthine.

6. The method of claim 4, wherein the nonspecific phosphodiesterase inhibitor is pentoxifylline.

7. A method for treating a disease, disorder or pathological condition in a mammalian subject in need thereof, comprising locally administering to the subject a pharmaceutical formulation consisting essentially of a therapeutic effective amount of a compound of formula I: 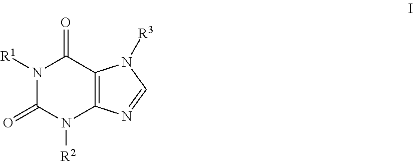
or a pharmaceutically acceptable salt thereof, wherein:
each of R¹, R² and R³ is independently selected from the group consisting of H, C₁-C₆ alkyl, C₂-C₆ alkenyl, C₂-C₆ alkynyl, cycloalkyl, heterocyclyl, aryl and heteroaryl, each of which is further optionally substituted,
with the further proviso that the disease, disorder or pathological condition is selected from the group consisting of Dupuytren's contracture, frozen shoulder, lipoma, cellulite, uterine fibroids, glaucoma, hyperthrophic scars, scarred tendons, keloids, herniated intervertebral disks and vitrectomy.

8. The method of claim 7, wherein each of R¹, R² and R³ is independently selected from the group consisting of H and an optionally substituted C₁-C₆ alkyl.

9. The method of claim 7, wherein each of R¹, R² and R³ is independently selected from the group consisting of H, a C₁-C₆ alkyl and an acyl-substituted C₁-C₆ alkyl.

10. The method of claim 7, wherein the compound of formula I is a nonspecific phosphodiesterase inhibitor.

11. The method of claim 10, wherein the nonspecific phosphodiesterase inhibitor is selected from the group consisting of pentoxifylline, caffeine, aminophylline, enprofylline, isbufylline, theophylline, theobromine and 3-isobutyl-1-methylxanthine.

12. The method of claim 11, wherein the nonspecific phosphodiesterase inhibitor is pentoxifylline.

13. The method of claim 1, wherein the disease, disorder or pathological condition is Dupuytren's contracture.

14. The method of claim 13, wherein the pharmaceutical formulation is injected into the hand or paw of the mammalian subject.

15. The method of claim 7, wherein the disease, disorder or pathological condition is Dupuytren's contracture.

16. The method of claim 15, wherein the pharmaceutical formulation is injected into the hand or paw of the mammalian subject.

17. The method of claim 1, wherein the therapeutic effective amount is between 4 mg and 20 mg.

18. The method of claim 1, wherein the pharmaceutical formulation further comprises at least one second active agent selected from the group consisting of alprostadil, papaverine, phentolamine nitrovasodilators, alpha receptor blocking agents, ergot alkaloids, antihypertensive agents, vasodilators, semisynthetic and synthetic prostaglandins, vasoactive intestinal peptides and a collagenase.
